(19) United States
(12) Patent Application Publication
(10) Pub. No.: US 2005/0169871 A1
(43) Pub. Date: Aug. 4, 2005

(54) Composition prepared with pressurized fluid, uses therefore

(75) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/033,118

(22) Filed: Jan. 12, 2005

Related U.S. Application Data
(60) Provisional application No. 60/560,260, filed on Apr. 8, 2004.

(30) Foreign Application Priority Data
Jan. 29, 2004 (FR) 0400853

(51) Int. Cl. A61K 7/06; A61K 7/11

(52) U.S. Cl. 424/70.15

(57) ABSTRACT

The invention relates to a process for preparing a cosmetic composition for the cosmetic treatment of keratin materials, wherein it comprises a step of percolating steam at a pressure of at least 3 bar through at least one anionic polymer and/or one nonionic polymer, in solid form.

### REFERENCE TO PRIOR APPLICATIONS

This application claims priority to U.S. provisional application 60/560,260 filed Apr. 8, 2004, and to French patent application 0400853 filed Jan. 29, 2004, both incorporated herein by reference.

### FIELD OF THE INVENTION

The present invention relates to a process for preparing a composition preferably useful for the cosmetic treatment of keratin materials, in particular the skin and human keratin fibres such as the hair, and also to the composition so prepared and to its uses in treating keratin materials.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The drawings and description are to be regarded as illustrative in nature, and not as restrictive.

### BACKGROUND OF THE INVENTION

In cosmetics, it is always sought to improve the cosmetic properties of keratin materials, and especially the styling properties of the hair. Cosmetic treatment compositions comprising styling agents such as anionic or nonionic polymers, which are intended to give the hair sheen, lightness, body and hold and to facilitate its disentangling, may thus be applied thereto.

However, cosmetic treatment compositions containing such styling agents are generally aqueous compositions in which the said agents must be dissolved. The lack of solubility of these styling agents limits the styling power of these compositions. In addition, this solubility criterion reduces the number of styling agents that may be used for the cosmetic treatment of keratin materials. Moreover, certain fixing polymers contain monomers with ester units or are polyesters derived from the polycondensation of diols and diacids. They are therefore compounds that are sensitive to hydrolysis, in particular in acidic or alkaline media, which limits their formulation possibilities.

### DETAILED DESCRIPTION OF THE PREFERRED EMBODIMENTS

It has now been discovered, surprisingly, that by using a novel process for preparing a composition, compositions that are more or less concentrated in styling agent(s) may be obtained in a very short time, for example of less than 2 minutes, according to need, especially without preserving agent, enabling the solubility and stability problems outlined above to be overcome.

This process may be performed simply. A pressurized fluid, the temperature of which is preferably greater than or equal to 30° C., preferably ranging from 30° C. to 150° C. and even more preferably ranging from 40° C. to 120° C., is passed for a very short time, of less than one minute, through at least one anionic polymer and/or one nonionic polymer in solid or pasty form, preferably in solid form and more preferably in pulverulent form.

It also enables the use in anhydrous form of anionic and/or nonionic polymers that are unstable in aqueous compositions either because they react with water or because they react in aqueous solution with compounds that do not react with them in an anhydrous composition.

The compositions prepared according to this process may have limited stability on storage, which is not a drawback in this case since the process leads to a ready-to-use composition intended to be used quickly after its preparation, for example within five minutes of preparation, especially after cooling to a cosmetically acceptable temperature, preferably below 60° C. The composition may be used up to one week or more after its preparation, depending on the rate of degradation of the anionic and/or nonionic polymer.

Given the very short preparation time, the invention compositions may be prepared “on demand” by mixing together various active compounds, according to the desired, e.g., cosmetic, properties.

According to another embodiment, the anionic and/or nonionic polymers may be packaged in a ready-to-use device, and it is not necessary to determine beforehand the concentrations of the active compounds in solution, which limits measuring errors by the user.

In addition, the process according to the invention makes it possible to avoid the use of multi-compartment bottles, which makes the process particularly economical and safer for the user.

The composition thus obtained may be used alone or as a mixture with another composition.

An important advantage of this preparation process is the production of compositions that give good cosmetic properties. In particular, keratin fibres treated with a composition obtained via the process according to the invention have improved styling properties, especially in terms of disentangling, sheen, lightness, improved visual appearance, body and hold.

One preferred subject of the invention is thus a process for preparing a cosmetic composition for treating keratin materials, wherein it comprises a step of percolating a fluid at a pressure of at least 3 bar through at least one anionic polymer and/or one nonionic polymer, in solid or pasty form.

Another subject of the invention is a composition obtained via the process according to the invention.

Another subject of the invention is the use of the composition obtained according to the process of the invention for the cosmetic treatment of keratin materials, and especially for styling the hair.

Another subject of the invention is a packaging device for performing the preparation process of the present invention.

Other subjects, characteristics, aspects and advantages of the invention will emerge even more clearly on reading the further description and the examples that follow.

According to the invention, the invention process comprises a step of percolating a fluid, preferably at a temperature of greater than or equal to 30° C., preferably ranging from 30° C. to 150° C. and better still ranging from 40° C. to 120° C., at a pressure of at least 3 bar (3×105 Pa), through at least one anionic polymer and/or one nonionic polymer in solid or pasty form.

Percolation is a movement of fluid through a saturated porous medium, allowing the passage of the fluid due to the action or effect of pressure.

The fluid may be steam optionally accompanied by liquid water, one or more cosmetically acceptable, especially organic, liquid and/or gaseous solvents, or alternatively of a mixture of steam optionally accompanied by liquid water, and of one or more cosmetically acceptable liquid and/or gaseous solvents. Preferably, the fluid comprises at least steam possibly accompanied by liquid water, and even more preferably it is steam possibly accompanied by liquid water.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, and mixtures thereof.

The term “water-insoluble compound” means any compound which, at a concentration of greater than or equal to 0.1% by weight in water at 25° C., does not form an isotropic solution that is transparent to the naked eye.

The anionic polymer and/or the nonionic polymer is in solid form or in pasty form, preferably in solid form and even more preferably in pulverulent form.

For the purposes of the present invention, the term “pasty form” means a consistency intermediate between a solid phase and a liquid phase. The viscosity of this pasty phase is preferably greater than 0.1 Pa.s and even more preferably greater than 1 Pa.s, at 25° C. with a shear rate of 10 s-1.

The term “keratin materials” includes the skin, mucous membranes such as the lips, and/or the integuments such as the nails and keratin fibres, for example the eyelashes, the eyebrows and the hair.

The process of the present invention may be performed using a standard device for generating a pressurized fluid, at a temperature preferably of greater than or equal to 30° C. and even more preferably ranging from 30° C. to 150° C. Such a device preferably comprises a pressure-resistant chamber, equipped with a thermal block, and also a circuit for conveying the fluid produced to the anionic and/or nonionic polymer.

In a preferred embodiment the device comprises therein a composition comprising at least one anionic polymer and/or one nonionic polymer, in solid or pasty form.

According to another embodiment, the device furthermore comprises a reservoir of liquid(s) and also a pump for conveying the liquid(s) to the chamber.

The liquid contained in the reservoir is either water, or a cosmetically acceptable solvent or a mixture of several cosmetically acceptable solvents, or alternatively a mixture of water and of one or more cosmetically acceptable solvents. Preferably the liquid comprises at least water, and even more preferably the liquid is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., preferably ranging from 30° C. to 150° C., under a pressure of at least 4 bar, preferably greater than 10 bar and most particularly between 10 and 30 bar.

According to one particular embodiment of the invention, the percolation step is performed with steam at a pressure between 3 and 30 bar, or of at least 4 bar, preferably greater than 10 bar and most particularly between 10 and 30 bar.

The anionic and/or nonionic polymer(s), in solid or pasty form, may be used directly in the device for generating the pressurized fluid in a container intended for this use. They may also be packaged in a particular device for packaging a cosmetic composition, comprising a closed housing delimited by at least one wall that is at least partially permeable to a fluid under a pressure of at least 3 bar, the composition containing at least one anionic polymer and/or one nonionic polymer. Such devices are described, for example, in patent applications WO 00/56629, EP 512 470 or WO 99/03753.

According to one particular embodiment, the housing is delimited by two sealed sheets. According to another embodiment, the housing is delimited by a tray closed with a lid.

These devices may be manufactured, for example, from woven or nonwoven plastic or plant materials, for example cellulose, metal such as aluminium, or composite materials. Such devices are described, for example, in patent applications WO 00/56629, EP 512 470 or WO 99/03753.

The anionic fixing polymers generally used are preferably polymers comprising groups derived from carboxylic acid, sulfonic acid or phosphoric acid and have a number average molecular weight of approximately between 500 and 5 000 000.

The carboxylic groups are provided by unsaturated monocarboxylic or dicarboxylic acid monomers such as those corresponding to the formula:

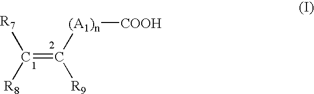

in which n is an integer from 0 to 10, A1 denotes a methylene group, optionally connected to the carbon atom of the unsaturated group, or to the neighbouring methylene group when n is greater than 1, via a hetero atom such as oxygen or sulfur, R7 denotes a hydrogen atom or a phenyl or benzyl group, R8 denotes a hydrogen atom or a lower alkyl or carboxyl group, R9 denotes a hydrogen atom, a lower alkyl group or a —CH2-COOH, phenyl or benzyl group.

In the abovementioned formula, a lower alkyl group preferably denotes a group having 1 to 4 carbon atoms and in particular methyl and ethyl.

The anionic fixing polymers containing carboxylic groups that are preferred according to the invention are:

A) acrylic or methacrylic acid homo- or copolymers, or salts thereof, copolymers of acrylic acid and of acrylamide, the sodium salts of polyhydroxycarboxylic acids.

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent 1 222 944. and German patent application 2 330 956, the copolymers of this type comprising an optionally N-alkylated and/or hydroxyalkylated acrylamide unit in their chain as described in particular in Luxembourg patent applications 75370 and 75371. Mention may also be made of copolymers of acrylic acid and of C1-C4 alkyl methacrylate and terpolymers of vinylpyrrolidone, of acrylic acid and of methacrylate of C1-C20 alkyl, for example of lauryl, and methacrylic acid/ethyl acrylate/tert-butyl acrylate terpolymers.

Mention may also be made of methacrylic acid/acrylic acid/ethyl acrylate/methyl methacrylate copolymers as an aqueous dispersion.

C) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted or crosslinked, or alternatively another vinyl, allylic or methallylic ester monomer of an α- or β-cyclic carboxylic acid. Such polymers are described, inter alia, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) copolymers derived from C4-C8 monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in particular in U.S. patents U.S. Pat. No. 2,047,398, U.S. Pat. No. 2,723,248 and U.S. Pat. No. 2,102,113 and GB patent GB 839 805;
copolymers comprising (i) one or more maleic, citraconic or itaconic anhydride units and (ii) one or more monomers chosen from allylic or methallylic esters optionally comprising one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

These polymers are described, for example, in French patents FR 2 350 384 and FR 2 357 241 by the Applicant.

E) polyacrylamides comprising carboxylate groups.

The homopolymers and copolymers comprising sulfonic groups are polymers comprising vinylsulfonic, styrenesulfonic, naphthalenesulfonic or acrylamido-alkylsulfonic units.

These polymers can be chosen in particular from:
polyvinylsulfonic acid salts having a molecular weight of approximately between 1000 and 100 000, as well as the copolymers with an unsaturated comonomer such as acrylic or methacrylic acids and their esters, as well as acrylamide or its derivatives, vinyl ethers and vinylpyrrolidone;
polystyrenesulfonic acid salts. These compounds are described in patent FR 2 198 719;
polyacrylamidesulfonic acid salts, such as those mentioned in patent U.S. Pat. No. 4,128,631 and more particularly polyacrylamidoethylpropanesulfonic acid.

Mention may also be made of nonionic polyurethanes.

According to the invention, the anionic fixing polymers are preferably chosen from acrylic acid copolymers, such as the acrylic acid/ethyl acrylate/N-tert-butylacrylamide terpolymers, copolymers derived from crotonic acid, such as vinyl acetate/vinyl tert-butylbenzoate/crotonic acid terpolymers and crotonic acid/vinyl acetate/vinyl neododecanoate terpolymers, polymers derived from maleic, fumaric or itaconic acids or anhydrides with vinyl esters, vinyl ethers, vinyl halides, phenylvinyl derivatives and acrylic acid and esters thereof, such as methyl vinyl ether/monoesterified maleic anhydride copolymers, copolymers of methacrylic acid and of methyl methacrylate, copolymers of methacrylic acid and of ethyl acrylate, vinyl acetate/crotonic acid copolymers and vinyl acetate/crotonic acid copolymers grafted with polyethylene glycol.

The nonionic fixing polymers that may be used according to the present invention are chosen, for example, from:
polyalkyloxazolines;
vinyl acetate homopolymers;
vinyl acetate copolymers, for instance copolymers of vinyl acetate and of acrylic ester, copolymers of vinyl acetate and of ethylene, or copolymers of vinyl acetate and of maleic ester, for example of dibutyl maleate;
acrylic ester homopolymers and copolymers, for instance copolymers of alkyl acrylates and of alkyl methacrylates;
copolymers of acrylonitrile and of a nonionic monomer chosen, for example, from butadiene and alkyl (meth)acrylates;
styrene homopolymers;
styrene copolymers, for instance copolymers of styrene and of alkyl (meth)acrylate; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, and
vinyllactam copolymers, poly(vinylpyrrolidone/vinyl acetate) copolymers and poly(vinylpyrrolidone/vinyl acetate/vinyl propionate) terpolymers;
nonionic polyurethanes.

The alkyl groups of the nonionic polymers mentioned above preferably contain from 1 to 6 carbon atoms.

The anionic and/or nonionic polymer(s) according to the invention may be used as a mixture with one or more solid or pasty, and preferably pulverulent, adjuvants. The adjuvants may be chosen from clays, salts, anionic, cationic, nonionic, amphoteric or zwitterionic surfactants, natural or synthetic thickeners, glass beads, silica, Nylon, alumina, titanium dioxide, zeolites, chitosan, maltodextrin, cyclodextrin, mono- or disaccharides, for instance glucose, sucrose, sorbitol or fructose, zinc oxide, zirconium oxide, resin particles, for instance silicone or silica beads, talc, borosilicates, especially calcium borosilicate, polyethylene, cotton, polytetrafluoroethylene (PTFE), cellulose and its derivatives, superabsorbent compounds, magnesium carbonate, calcium carbonate, optionally modified starch corn seeds, polydimethylsiloxane gums, porous hydroxyapatite, silk, collagen, sawdust, wrack powder, meals or extracts of wheat, rice, pea, lupin, soybean or barley, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the anionic and/or nonionic polymer(s) of the invention is (are) preferably in an amount ranging from 0.5% to 99% by weight, better still from 1% to 80% by weight and even more preferably from 2% to 60% by weight relative to the total weight of anionic and/or nonionic polymer(s) and adjuvants.

The plants or a plant extracts used may be subjected, prior to percolation, to a treatment such as roasting, cryogrinding or lyophilization.

The cosmetic composition for treating keratin materials obtained according to the process of the invention may contain, besides the anionic and/or nonionic polymer(s) and the component(s) of the fluid, i.e. water and/or cosmetically acceptable solvent(s), optionally all or some of the adjuvants(s) present in the solid or pasty mixture.

The invention also relates to a composition that may be obtained via the process according to the invention, the composition preferably being free of preserving agents.

Using the preparation process of the invention, a cosmetic composition for treating keratin materials is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The cosmetic composition for treating keratin materials optionally resulting from the mixture(s) and/or addition(s) indicated above will be referred to hereinbelow as the final cosmetic treatment composition or final composition.

One particular mode of implementation of the invention is applying the composition obtained by means of a device not requiring any human intervention, and optionally equipped with a cooling means.

The amount of the anionic and/or nonionic polymer(s) present in the final cosmetic treatment composition obtained via the process of the present invention is generally preferably between 0.001% and 50% by weight approximately, more preferably between 0.005% and 30% and even more preferably between 0.01% and 20%, relative to the total weight of the final cosmetic treatment composition.

When the composition obtained via the process of the present invention is mixed with a cosmetically acceptable medium, the medium generally contains water or a mixture of water and of at least one organic solvent to dissolve the compounds that would not be sufficiently soluble in water.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether and diethylene glycol monomethyl ether and monoethyl ether, and also aromatic alcohols, for instance benzyl alcohol or phenoxyethanol, and mixtures thereof.

The solvents are preferably present in proportions preferably of between 1% and 40% by weight and even more preferably between 5% and 30% by weight relative to the total weight of the final composition.

At least one additive, for example those conventionally used in cosmetics, may also be added to the cosmetic treatment compositions obtained according to the process of the present invention. Examples of such additives that may be mentioned include anionic, cationic, nonionic, amphoteric or zwitterionic surfactants, or mixtures thereof, cationic, amphoteric or zwitterionic polymers, or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioning agents, such as, for example, silicone oils, film-forming agents, ceramides, preserving agents and opacifiers, and also oils, waxes, gums and coloured or nacreous pigments.

The above additives are generally present in an amount for each of them of between 0.01% and 20% by weight relative to the weight of the final composition.

Needless to say, a person skilled in the art will take care to select this or these optional additional compounds such that the advantageous properties intrinsically associated with the cosmetic treatment composition in accordance with the invention are not, or are not substantially, adversely affected by the addition(s) envisaged.

The pH of the final composition is preferably generally between about 3 and 12 and more preferably between about 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as mono-, di- and triethanolamine and derivatives thereof, sodium hydroxide, potassium hydroxide and the compounds of formula (II) below:

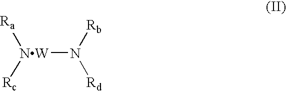

in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The invention compositons, including the final cosmetic treatment composition, may be in any various form, such as in the form of liquids, creams, mascara or gels, or in any other form, and preferably in a form that is suitable for treating keratin materials, and especially keratin fibres, and the skin.

The invention compositons, including the final cosmetic treatment composition, may be used, for example, as a shampoo, a rinse-out or leave-in conditioner, a deep-down care mask, a shower gel, or a lotion or cream for treating keratin materials.

The present invention also relates to a process for treating keratin materials, comprising the preparation of a composition according to the process as defined above, and its application to the keratin materials, for example by means of an operator or by means of a device not requiring any human intervention. The application time may range between 15 seconds and one hour.

Before application, the composition obtained according to the process of the invention may be mixed with a cosmetically acceptable medium and/or with one or more additives conventionally used in cosmetics, as described above.

Another embodiment is preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The examples below are intended to illustrate the present invention, but not limit it.

### EXAMPLE 1

The ingredients below are mixed together in the proportions indicated as weight percentages relative to the total weight of solid mixture:

| | |
|---|---|
| vinylpyrrolidone/vinyl acetate (65/65) copolymers sold | 70% |
| under the trade name PVP/VA S 630 L by the company ISP | |
| cetylhydroxyethylcellulose sold under the trade name | 30% |
| Natrosol Plus Grade 330 by the company Aqualon | |

5 g of this mixture are placed in a commercial espresso machine. Steam is then passed through until a composition (A) having a final volume of 50 ml is obtained.

One part by weight of an aqueous composition (B) containing 1% by weight of hydroxyethylcellulose may then be added to two parts by weight of composition (A), to facilitate the application.

A cosmetic treatment composition ready to be applied to the hair is thus obtained.

Light, shiny hair that looks pleasant and is easy to disentangle is thus obtained.

### EXAMPLE 2

The ingredients below are mixed together in the proportions indicated as weight percentages relative to the total weight of solid mixture:

| | |
|---|---|
| polydimethyl/methylsiloxane containing 3-propylthio | 40% |
| methyl acrylate/methyl methacrylate/methacrylic acid groups, | |
| sold under the trade name VS 80 Dry by the company 3M | |
| sodium carboxymethyl cellulose sold under the trade name | 45% |
| Blanose 7M8SF by the company Aqualon | |
| D-xylose | 10% |
| cetylhydroxyethylcellulose sold under the trade name | 5% |
| Natrosol Plus Grade 330 by the company Aqualon | |

5 g of this mixture are placed in a commercial espresso machine. Steam is then passed through until a composition (A) having a final volume of 50 ml is obtained.

One part by weight of an aqueous composition (B) containing 1% by weight of hydroxyethylcellulose may then be added to two parts by weight of composition (A), to facilitate the application.

A cosmetic treatment composition ready to be applied to the hair is thus obtained.

Light, shiny hair that looks pleasant and is easy to disentangle is thus obtained.

The above written description of the invention provides a manner and process of making and using it such that any person skilled in this art is enabled to make and use the same, this enablement being provided in particular for the subject matter of the appended claims, which make up a part of the original description and including a process for preparing a composition, wherein it comprises a step of percolating a fluid at a pressure of at least 3 bar through at least one anionic polymer and/or one nonionic polymer, in solid or pasty form. Preferred embodiments of the invention similarly fully described and enabled include the composition so made and the use of a composition obtained via the process according to the invention for the treatment of keratin materials.

As used herein, the phrases “selected from,” “selected from the group consisting of,” “chosen from,” and the like include mixtures of the specified materials.

Where compounds are described as, e.g., “ . . . cellulose and its derivatives . . . ” an alternate is “cellulose and cellulose compounds,” where both cellulose derivatives and cellulose compounds are understood to share a common core/structure with cellulose. Those of ordinary skill are able to identify “X” derivatives based on a knowledge of “X” and the structural similarity of the compounds.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid at a pressure of at least 3 bar through at least one anionic polymer and/or one nonionic polymer, in solid or pasty form.

2. A process according to claim 1, comprising percolating a fluid at a pressure of at least 3 bar through at least one anionic polymer, wherein the anionic polymer comprises groups derived from carboxylic acids, sulfonic acids or phosphoric acids and having a weight-average molecular weight of between 500 and 5 000 000, and mixtures thereof.

3. A process according to claim 1, comprising percolating a fluid at a pressure of at least 3 bar through at least one nonionic polymer, wherein the nonionic polymer is selected from vinylpyrrolidone homopolymers, copolymers of vinylpyrrolidone and of vinyl acetate, polyalkyloxazolines, vinyl acetate homopolymers, copolymers of vinyl acetate and of acrylic ester, copolymers of vinyl acetate and of ethylene, copolymers of vinyl acetate and of maleic ester, copolymers of polyethylene and of maleic anhydride, alkyl acrylate homopolymers and alkyl methacrylate homopolymers, acrylic ester copolymers, copolymers of acrylonitrile and of butadiene or of alkyl (meth)acrylate, polyurethanes, copolymers of alkyl acetate and of urethane, and polyamides, and mixtures thereof.

4. A process according to claim 1, wherein the anionic and/or nonionic polymer(s) in solid or pasty form is (are) present in a mixture with at least one adjuvant.

5. A process according to claim 4, wherein the adjuvant is selected from clays, salts, anionic, nonionic, cationic or zwitterionic surfactants, natural or synthetic thickeners, starch, optionally modified, glass beads, silica, Nylon, alumina, titanium dioxide, zeolites, chitosan, maltodextrin, cyclodextrin, mono- or disaccharides, zinc oxide, zirconium oxide, resin particles, talc, borosilicates, polyethylene, cotton, polytetrafluoroethylene (PTFE), cellulose and its derivatives, superabsorbent compounds, magnesium carbonate, calcium carbonate, corn seeds, polydimethylsiloxane gums, porous hydroxyapatite, silk, collagen, sawdust, wrack powder, meals or extracts of wheat, rice, pea, lupin, soybean or barley, calcium alginate, active charcoal, poly(vinylidene chloride/acrylonitrile) particles, and mixtures thereof.

6. A process according to claim 4, wherein the anionic and/or nonionic polymer(s) is (are) present in an amount of 0.5% to 99% by weight relative to the total weight of anionic and/or nonionic polymer(s) and adjuvant.

7. A process according to claim 1, wherein the percolation step is performed with a fluid at a pressure of between 3 and 30 bar.

8. A process according to claim 1, wherein the percolation step is performed with a fluid at a pressure of at least 10 bar.

9. A process according to claim 1, wherein the fluid is steam, optionally accompanied by liquid water.

10. A process according to claim 1, wherein the fluid conprises one or more cosmetically acceptable liquid and/or gaseous organic solvents.

11. A composition obtained by a process comprising percolating a fluid at a pressure of at least 3 bar through at least one anionic polymer and/or one nonionic polymer, in solid or pasty form.

12. The composition according to claim 11, wherein said compositon is free of preserving agent.

13. A process for treating keratin material, comprising applying the composition of claim 11 to the keratin material.

14. The process according to claim 13, wherein the composition is applied to the keratin material by a device not requiring any human intervention.

15. The process according to claim 13, wherein, before application, the composition is mixed with a cosmetically acceptable medium and/or with one or more additives.

16. The process according to claim 13, wherein, before application, the composition is mixed with a different composition obtained by a process comprising percolating a fluid at a pressure of at least 3 bar through at least one anionic polymer and/or one nonionic polymer, in solid or pasty form.

17. The process according to claim 13, wherein said process is a process for providing improved styling properties to keratin material.

18. The process according to claim 13, wherein said process provides said keratin material with at least one of disentanglement, sheen, lightness, improved visual appearance, body and hold.

19. A device for packaging a cosmetic composition, comprising a closed housing delimited by at least one wall that is at least partially permeable to a fluid at a pressure of at least 3 bar, the device comprising therein a composition comprising at least one anionic polymer and/or one nonionic polymer, in solid or pasty form.

20. Device according to claim 19, wherein the housing is delimited by two sealed sheets, or by a tray closed with a lid.
